(19) United States
(12) Patent Application Publication
(10) Pub. No.: US 2002/0187485 A1
(43) Pub. Date: Dec. 12, 2002

(54) Open substrate platforms suitable for analysis of biomolecules

(76) Inventors: Mogens Hausteen Jakobsen, Vanlose (DK); Lars Kongsbak, Holte (DK)

Correspondence Address: Edwards & Angell, LLP, P.O. Box 9169, Boston, MA, 02209, US

(21) Appl. No.: 10/032,301

(22) Filed: Oct. 25, 2001

Related U.S. Application Data
(60) Provisional application No. 60/243,349, filed on Oct. 25, 2000.
(60) Provisional application No. 60/305,726, filed on Jul. 16, 2001.

(51) Int. Cl. C12Q 1/68; G01N 1/30; G01N 33/48; C12M 1/34; C12M 1/22

(52) U.S. Cl. 435/6; 435/40.5; 435/287.2; 435/305.1

(57) ABSTRACT

The invention relates to novel analysis slides or substrates of the open configuration. The slides may be used for any application which normally utilizes a conventional microscope slide and can be used in conjunction with any type of equipment typically used to manipulate or evaluate a standard microscope slide. In particular, the invention provides open slides for covalent immobilization of polypeptides and nucleic acids. Further provided are methods for carrying out biological assays using arrays of biomolecules immobilized on the slides of the invention.

The present application claimed the benefit of U.S. provisional application No. 60/243,349 filed Oct. 25, 2000, and U.S. provisional application No. 60/305,726 filed Jul. 16, 2001, both incorporated herein by reference in their entirety.

### BACKGROUND OF THE INVENTION

1. Field of the Invention.

The invention relates to novel platforms, particularly slides and compartments such as microscopic slides, of the open configuration. The slides may be used for any application which normally utilizes a conventional microscope slide and can be used in conjunction with any type of equipment typically used to manipulate or evaluate a standard microscope slide. In particular, the invention provides open slides for covalent immobilization of biomolecules, e.g. peptides, polypeptides, nucleic acids, nucleic acid binding partners, proteins, receptors, antibodies, enzymes, oligo saccharides, polysaccharides, cells, arrays of ligands (e.g. non-protein ligands), and the like. Further provided are methods for carrying out biological assays using arrays of biomolecules immobilized on the slides of the invention.

2. Background.

The development of bio-array technologies promises to revolutionize the way biological research is carried out. Bio-arrays, wherein a library of biomolecules is immobilized on a small slide or chip, allow hundreds or thousands of assays to be carried out simultaneously on a miniaturised scale. This permits researchers to quickly gain large amounts of information from a single sample. In many cases, bio-array type analysis would be impossible using traditional biological techniques due to the rarity of the sample being tested and the time and expense necessary to carry out such a large scale analysis.

Although bio-arrays are powerful research tools, they suffer from a number of shortcomings. For example, bio-arrays tend to be expensive to produce due to difficulties involved in reproducibly manufacturing high quality arrays. Also, bio-array techniques can not always provide the sensitivity necessary to perform a desired experiment. Therefore, it would be desirable to provide an improved platform for the production of arrays which results in a less expensive, more reproducible and more sensitive bio-array.

There are two fundamentally different approaches to the manufacturing of bio-arrays: 1) “in situ synthesis” and 2) “micro spotting”. The in situ synthesis approach involves monomer-by-monomer synthesis directly on the substrate carrier. This approach has some inherent drawbacks as the synthesis of oligomers includes many chemical steps which never provide 100% yield. Thus, bio-arrays produced via the in situ synthesis strategy generally contain truncated sequences leading to differences in the composition from array to array. The micro spotting approach involves dispensing of biomolecules onto the substrate carrier followed by immobilization of the molecules onto the surface. This approach offers the advantage that materials can be obtained from natural sources, or synthesized on standard synthesizers, purified and characterized prior to construction of the array. Thus, bio-arrays produced by the micro spotting approach generally are more reproducible and of higher quality than bio-arrays produced by the in situ synthesis approach.

### SUMMARY OF THE INVENTION

The present invention provides novel substrate analysis platforms that can be employed in a variety of scanning or analysis apparatus, including applications or instruments which normally employs a standard microscope slide. A preferred use of the platforms is the immobilization of biomolecules for investigation of biomolecule interactions.

In a first embodiment, a slide article, preferably rectangular and plastic, and comprised of at least one or more shallow depressions on the top surfaces and at least one depression on the bottom surface, is provided. The depression(s) on the top surface provides a well capable of containing a specific volume of liquid. The depression on the bottom surface prevents the slide from becoming scratched during handling. The slide preferably contains paired finger indentations to aid in removal of the slide from a flat surface. The slide is preferably used in conjunction with a coverslip which is capable of sealing the opening of the well on the top surface of the slide due to hydrophilic interactions.

The slides are preferably constructed of a polymer with low intrinsic fluorescence emission. Preferably the polymer is resistant to extremes of temperature (high and low), sonication and a wide variety of solvent conditions, such as extremes of pH, high ionic strength or organic solvents. Preferred polymers include polycarbonate, Topas (tradename; available from Hoeschst). Other suitable materials of constructions of the analysis platforms of the invention include e.g. plastics, polyethylene, polypropylene, polystyrene, polymethylacrylate, and the like.

Slides of the invention may be used for any type of application which may be carried out using a standard microscope slide. For example, the slides may be used for microscopic analysis of samples, smears, sections, etc. Other types of applications include e.g. diagnostics; SNP analysis; gene expression including e.g. detection of intron/exon splicing, and to evaluate if expression of certain genes is modulated by drug candidates); toxicology studies including toxicology on cells; protein-to-protein interactions; plant and animal breeding studies; environmental studies; and the like.

Slides or analysis platforms of the invention may be suitably used in conjunction with any type of a wide variety of analysis equipment, materials or reagents, including equipment, materials and reagents used with standard microscope slides, such as e.g. coverslips, slide washers, pipettors, inkjet printers or spotters, or robotics systems. Additionally, the slides or analysis platforms of the invention may be analysed using any type of instrument or device capable of analysing or reading a standard microscope slide including, for example, microscopes, scanners, readers, imagers, or the like.

The invention also provides immobilized biomolecules on the surface of the substrate. Preferably, nucleic acid, nucleic acid binding partners, proteins, antibodies, polysaccharides or polypeptides are immobilized in an array wherein each unique sequence is located at a defined position on the substrate. The arrays preferably contain at least about 100 unique sequences per cm². Immobilized nucleic acids preferably contain from about 2 to about 5000 nucleotides, more typically 2 to about 1000 nucleotides, and polypeptides preferably contain from about 2 to about 5000 amino acids.

Immobilized nucleic acid chains of the invention preferably contain at least one LNA nucleoside analogue. LNA nucleoside analogues are disclosed in WO 99/14226. Also provided are oligomers composed entirely of LNA nucleosides. Immobilized nucleic acids may be either single stranded or double stranded.

Biomolecules are preferably immobilized onto the substrate using a photochemical linker, preferably a photoreactive linker, such as a photoreactive ketone, or particularly a photoreactive quinone such as disclosed in WO 96/31557. Also provided are flexible linkers which can serve as a spacer between the substrate surface and the biomolecule. Nucleic acid, polysaccharide and polypeptide chains are preferably immobilized via one end of the chain.

The invention also provides methods for carrying out biological assays using the substrate platforms and fluidic devices of the invention. A wide variety of assays may be carried on the analysis platforms and fluidic devices of the invention, including any type of assay which may be carried out using a standard microscope slide.

Specific examples include assays wherein one component is immobilized on the surface of the slide. Preferred assays involve immobilized arrays of polypeptide or nucleic acid sequences which may be exposed to a biomolecule (i.e. a nucleic acid, polypeptide, hormone, small molecule drug or drug candidate, etc.) under conditions which favor interaction between the biomolecule and the immobilized molecules. Preferably, interactions between the molecules are detected by virtue of a detectable feature on the biomolecule, e.g. a chemoluminescent tag such as a radiolabel (e.g. ¹²⁵I, tritium ³²P, ⁹⁹Tc, and the like); fluorescent tag; or an inducible tag e.g. a functional group that is activated by energy input such as electric impulse, radiation (e.g. UV radiation); and the like. The methods of the invention may be used e.g. to investigate interactions between nucleic acid-nucleic acid, nucleic acid-polypeptide, polypeptide-polypeptide, etc. Particularly preferred assays which may be performed using the methods of the invention include gene expression profiling; immunoassays; diagnostics; SNP analysis; gene expression including e.g. detection of intron/exon splicing, and the like.

Slides or analysis platforms of the invention may also be used for applications or assays not involving immobilized biomolecules.

Other aspects of the invention are disclosed infra.

### BRIEF DESCRIPTION OF THE DRAWINGS

FIG. 1 shows a plan view of the preferred embodiment of the open substrate platform for immobilization of biomolecules.

FIG. 2 shows a lengthwise cross-sectional view of the open substrate platform as shown in FIG. 1.

FIG. 3 shows a further cross-sectional view of the open substrate platform as shown in FIG. 1.

FIG. 4 shows a widthwise cross-sectional view of the open substrate platform as shown in FIG. 1.

FIG. 5 shows a cross-sectional view of the of the open substrate platform as shown in FIG. 1 detailing the recessed wells on the top and bottom sides of the slide.

FIG. 6 shows a plan view of a general form of the open substrate platform for immobilization of biomolecules comprising an inlet port.

### DETAILED DESCRIPTION OF THE INVENTION

The present invention provides open substrate platforms which are a significant improvement over standard microscope slides. The substrate platforms are preferably used for the immobilization of biomolecules, but may be used for any application normally utilizing a microscope slide.

As used herein the term “substrate platform”, “analysis platform”, or “slide element” or similar term refers to the foundation upon which biomolecules may be immobilized, samples may be applied for analysis or biological assays may be carried out. The terms “substrate platform”, “fluidic device”, “analysis platform”, “slide element” and ‘slide’ or “microscope slide” may be used interchangeably, however, where applicable, the term substrate platform refers to the part of the slide to which the sample is applied and the term slide refers to the entire structure including the substrate platform.

As used herein the term “microscope slide” or “standard microscope slide” refers to any type of slide which falls within the parameters recognized in the art. For example, in the United States, typical slide elements have dimensions of 1 inch×3 inches. In Europe, typical slide dimensions include 25 mm×75 mm, or 26 mm×76 mm. Typical slide thickness are from about 1 mm to about 1.3 mm.

The substrate platform may be constructed from a variety of materials such as plastics, quartz, silicon, polymers, gels, resins, carbon, metal, membranes, glass, etc. or from a combination of several types of materials such as a polymer blend, polymer coated glass, silicon oxide coated metal, etc. Particularly preferred substrate materials are polymers which contain a low intrinsic fluorescence emission, such as polycarbonate, Topas (tradename; available from Hoechst), polymethylmethacrylate (PMMA), and the like.

The term “plastics” as used herein refers to polymers, such as thermoplastic polymers. The plastic is used in the manufacture of microfluidic devices. Such devices include, but are not limited to: miniature diagnostic systems for biopharmaceutical applications, miniature devices for directing fluid flow, miniature sensor devices for pharmaceutical and biochemical applications, and three-dimensional microfluidic systems. When used in these applications, it is preferred that the plastic is selected from the group consisting of homopolymers and copolymers of polycarbonate, polystyrene, polyacrylic, polyester, polyolefin, polyacrylate, and mixtures thereof.

The term “low intrinsic fluorescence” as used herein refers to a material or substrate which emits less than about 50 percent of the detected signal of a test sample on the substrate, thereby providing a signal:noise ratio at detection levels of 2:1.

The term “clarity” as used herein, is the degree of absence of impurities which may impair the passage of light through the slide and is measured by the amount of light that can pass through the slide, measured at a wavelength of preferably 530 nm. The amount of light passing through the slide is preferably at least 75% of total light from the light source, more preferably 85%, most preferably 90%.

Preferably, the substrate platform is constructed of a material that is capable of covalently binding to a biomolecule without activating the surface of the platform. For example, the substrate material may provide reactive groups at the surface such as carboxyl, amino, hydroxyl, sulfhydryl, etc. Alternatively, the surface of the substrate may be derivatized so as to provide functional groups which will allow covalent attachment of a biomolecule. For example, the substrate may be derivatized with silanes or other chemical groups; or the substrate may be surface modified such as by plasma treatment and the like; etc.

Preferably the surface of the substrate platform is substantially smooth so as to allow uniform binding of biomolecules and effective analysis of molecules bound to the substrate using a variety of scanners, readers, detectors, etc. Alternatively, the surface of the substrate may be treated or coated so as to increase the binding capacity of the substrate. For example, a greater surface area for biomolecule binding may be achieved by roughening the surface of the substrate or by coating it with gel, particles, beads, etc. Preferably the substrate platform is optimized so as to provide the greatest binding capacity while still allowing efficient manipulation and evaluation of biomolecules bound to the surface.

As used herein, the term “depression” refers to an indentation on the surface of the substrate analysis platform, wherein the indentation can be square or rectangular and the sides of the indented portion are either perpendicular to the indented surface or angled by at least 50° relative to the indented surface.

Particularly preferred are slides that have a flatness of less than or equal to about 20 μm, wherein the flatness does not deviate on a slide and between slides, more than 1 μm per millimeter. Preferably the slide has a roughness of about an RA of less than about 100 nm, preferably an RA of less than about 50 nm, more preferably an RA of less than about 20 nm.

The substrate platform is preferably constructed of materials which are resistant to extremes of low and high temperatures, i.e. temperatures of −5° C. to +105° C.; resistant to extremes of low and high pH, i.e. pH over a range of 1 to 13; resistant to sonication; and resistant to a wide variety of solvent conditions, i.e. high ionic strength and organic solvents such as ethanol, methanol, formamide, DMSO, etc. Particularly preferred substrate platforms are resistant to thermocycling such as performed during PCR. The substrate platforms are preferably resistant to multiple, i.e. about 10 to about 50 rounds of heating and cooling, such as would be obtainable with an art recognized thermocycler.

By the term ‘resistant’ it is meant that the fundamental shape and properties of the substrate platform are not altered in a way which will affect the performance or functionality of the platform. For example, resistance is meant to indicate that exposure to an extreme temperature or pH will not cause the platform to melt, warp, etc. and that the platform will still be capable of covalently binding a biomolecule to the surface after such exposure.

The substrate platform may be constructed in a variety of shapes and sizes so as to allow easy manipulation of the substrate and compatibility with a variety of standard lab equipment such as microtiter plates, multichannel pipettors, microscopes, inkjet-type array spotters, photolithographic array synthesis equipment, array scanners or readers, fluorescence detectors, infra-red (IR) detectors, mass spectrometers, thermocyclers, high throughput machinery, robotics, etc. For example, the substrate platform may be constructed so as to have any convenient shape such as a square, rectangle, circle, sphere, disc, slide, chip, film, plate, pad, tube or channel, strand, box, etc.

Preferably, the substrate platform is substantially flat with optional raised, depressed or indented regions to allow ease of manipulation. For example, the edges of the substrate platform may contain finger indents or ridges to facilitate handling and/or the surface may contain one or more wells which are capable of containing a specific volume of fluid. It is preferred that the substrate platforms have at least one depression on the bottom surface, the advantages being that depression(s) provide protection from scratching during handling; the substrate platform can be placed on a table or any work surface with a minimum risk of scratching; ease of stacking the slide for transport without the risk of a superadjacent slide being scratched by slides stacked above or below; ease of removing a wet slide from a surface without the problem of sticking to the surface due to capillary forces. Particularly preferred substrate platforms are constructed in the general size and shape of a microscope slide and are compatible with any type of instrument that is capable of manipulating or evaluating a microscope slide.

The substrate platform may contain one or more typically a plurality of channels or tubular sections that provide for flow and residence of test samples. For instance, configuration systems of the invention suitably may have flow channels for transport and analysis of a test sample. The substrate platform also typically has one, or a plurality of analytical areas. Such distinct analytical areas may reside e.g. in a test area of an open system of the invention, where each area is defined by a defined line, channel or the like in the substrate platform surface.

The substrate platform may be constructed in a variety of colors or with a variety of markings which perform both decorative and/or functional purposes. For example, the substrate platform may be constructed of materials containing dyes or pigments to provide a colored product. The color can serve as a means of identification or may serve to reduce the intrinsic fluorescence of the substrate material. Additionally, the substrate may be clear or opaque. Preferably, the substrate material is clear so as to allow light to pass through the substrate platform. In another aspect of the invention, the substrate platform may contain markings such as numbers, words, pictures, company logos, etc. In a particularly preferred embodiment, the substrate platform contains a bar code to allow unique identification of individual platforms.

Markings on the substrate platform may be made by any art recognized method including, for example, application of stickers or other adhesives; application of ink directly onto the substrate surface by a well-defined deposit e.g. an inkjet printer, a pin-spotter, etc.; raised or indented regions formed during the molding of the substrate platform; etched or frosted areas added after molding of the substrate platform; etc. Preferably, the markings are located outside the area to be used for sample analysis and may serve to demarcate the sample analysis area.

The substrate platforms of the invention may be constructed by any of a variety of methods, e.g. injection molding, hot embossing, mechanical machining, etching, with injection molding being generally preferred.

Substrate platforms of the invention may be constructed in an open configuration. By ‘open configuration’ it is meant that the substrate is not enclosed within a sealed container. Open platforms are preferably used in combination with covers and humidity chambers.

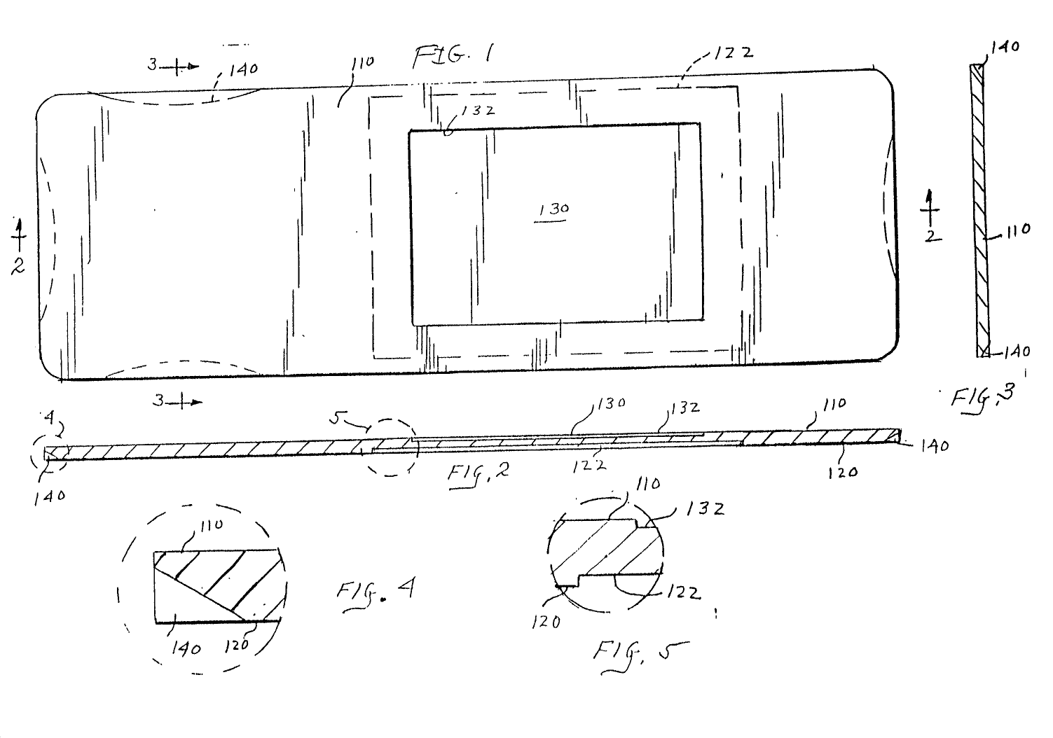

In a first embodiment of the invention, a rectangular, open, plastic substrate platform with the general dimensions of a microscope slide is provided as shown in FIGS. 1-5. The open slide 110 may be constructed from any polymer which contains an acceptable level of intrinsic background fluorescence. Particularly preferred materials are polycarbonate and Topas (tradename; available from Hoechst).

The open slide is preferably dimensioned so as to fit into any instrument or device which is capable of receiving a standard microscope slide. Specifically, the open slide is preferably from about 20 to about 30 mm wide, from about 70 to about 80 mm long and from about 0.1 to about 2 mm thick. More specifically, the open slide is preferably about 25 mm wide by 76 mm long by 1 mm thick. The top side of the slide 110 contains a defined region for covalent attachment of biomolecules referred to as the ‘analysis area’ 130. The analysis area is preferably from about 15 to about 22 mm wide and from about 20 to about 30 mm long. Most preferably, the analysis area is about 19 mm wide by about 28 mm long.

Preferred open substrate platforms of the invention comprise:

a slide element having opposing top and bottom surfaces, the slide element preferably being substantially rectangular and formed from a plastic material, and

wherein the top surface of the slide contains one or more depressions, preferably shallow depressions, with a defined area for sample analysis, and

wherein the bottom surface of the slide contains one or more depressions, preferably shallow depressions, opposing the depression on the top surface, and

preferably wherein the bottom surface of the slide further comprises at least one set of paired finger indentations for use in removing the slide from a flat surface.

Other preferred open substrate platforms of the invention comprise:

a slide element having opposing top and bottom surfaces, the slide element preferably being substantially rectangular and formed from a plastic material, and

wherein the top surface of the slide is comprised of a defined area for sample analysis, and

wherein the bottom surface of the slide contains one or more depressions, preferably shallow depressions, and

preferably wherein the bottom surface of the slide further comprises at least one set of paired finger indentations for use in removing the slide from a flat surface.

The open substrate platforms are suitably used in an array format, i.e. where multiple test samples are analyzed substantially simultaneously on the substrate platform. As referred to herein, the term “array” indicates a plurality of analytical data points that can be identified and address by their location in two or three-dimensional space, where i.e. identify can be established by the data point physical address.

Typically, the analysis systems of the invention utilize test samples that are in fluid form. For instance, test samples derived from humans or other mammals, or plant sample, may originate from blood, urine, or solid tissue or cells and will suitably be pre-treated to enrich or dilute the material to provide an optimized test sample.

In preferred analysis systems of the invention, the system will hold an accurate and reproducible volume of test sample fluid, e.g. in an open system, a volume of about 20 μl to about 30 μl is preferred, although other volumes also can be employed if desired.

As discussed above, analysis systems of the invention may have a relatively wide variety of dimensions. In one particularly preferred open system, the platform has outer dimensions of 25 mm×76 mm×1 mm. A preferred analytical area of that system will be 19 mm×28 mm and capable of holding a specific volume of fluid sample. A coverslip can be employed with the slide, preferably having the same or a different hydrophilicity than the analytical area to promote a robust sealing of the analytical area. The analytical area is designed, as described supra, so that placing the coverslip over the sample for analysis, does not bind to the sample or interfere with the sample in any way.

FIG. 1 of the drawings shows a plan view of a preferred embodiment of an open slide substrate platform. The open slide 100 is preferably constructed so as to contain shallow wells or depressions on the top 110 and/or bottom side 120 of the slide. The well on the top side of the slide 132 is constructed so as to be the same size or slightly larger than the analysis area 130. The well is preferably about 5 to about 100 μM deep, more preferably about 50 μM deep, and is capable of containing a precise volume of fluid. The well or depression on the bottom side of the slide 122 is constructed so as to be the same size or slightly larger than the well 132 on the top side of the slide. The well is preferably about 5 to about 250 μm deep, more preferably about 100 μm deep, and prevents the back side of slide corresponding to the analysis area from being scratched during routine handling. FIG. 5 shows a cross-sectional view detailing the top 132 and bottom 122 wells of the open slide as depicted in FIG. 1.

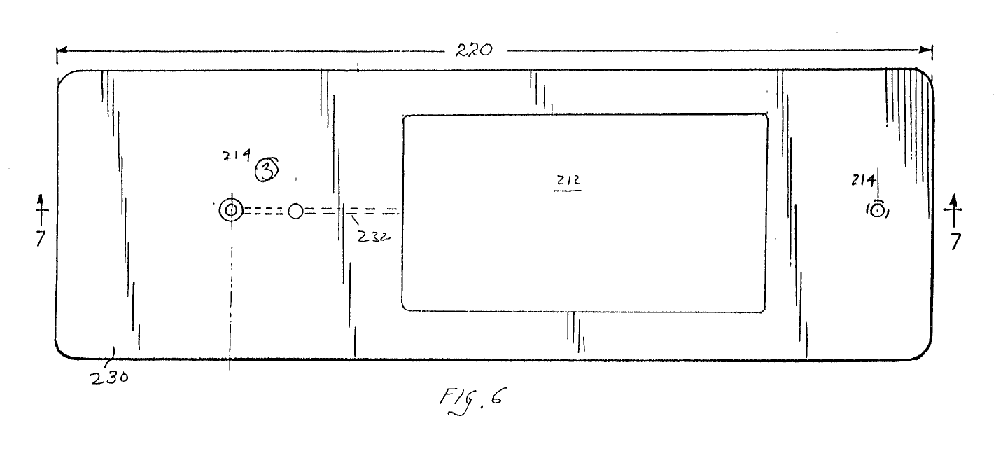

The open slide substrate platform is preferably constructed so as to contain finger indentations or contours 140. The finger indentations may be configured in a variety of styles or locations, but are preferably formed as semi-circular depression on the bottom side of the slide 120 (i.e. the side opposite the analysis area) so as to facilitate handling and removal from a flat surface. More preferably, pairs of finger indentations are located on opposite lengthwise and/or widthwise sides of the rectangular slide. FIG. 2 shows a lengthwise cross-sectional view of the open slide detailing a pair of finger indentations 140 on opposite lengthwise sides. FIG. 3 shows a cross-sectional view of the open slide detailing a pair of finger indentations 140 on opposite widthwise sides. FIG. 4 shows a detailed cross-sectional view of a finger indentation 140 as shown in FIG. 2.

The open slide is preferably used with a covering device or coverslip. The coverslip may be constructed of glass or plastic and is preferably clear so as to allow analysis of biomolecules bound to the analysis area. The coverslip is preferably thin, flat and dimensioned so as to be slightly larger than the well 132 on the top side of the slide. Preferably the coverslip is constructed of a material which has the same hydrophilicity or can be more hydrophilic than the surface of the slide so as to permit the coverslip to become sealed to the slide via a thin layer of aqueous solution. The coverslip permits the slide to be manipulated without loss of fluid due to spills or evaporation.

The open slide is preferably constructed using standard injection molding techniques, or other methods as discussed above. The marks left by the pin ejectors for extruding the slide from the mold are preferably located so as to be outside the analysis area 130.

The open substrate platform may also be preferably comprised of inlet ports for sample loading, buffer washing and air expulsion upon washing or loading. The inlet ports may be arranged in a variety of configurations so as to allow sample loading and washing without contamination of the analysis area. The sample ports are preferably funnel shaped with the wide end of the funnel toward the outside of the casing and the narrow end toward the inside of the casing, in order to facilitate introduction of liquid into the closed slide.

Preferably the sample and buffer ports may be configured so as to receive liquid from a variety of sources such as a pipette tip, a syringe, a tube or channel, a robotics system, etc. In a particularly preferred embodiment, the ports are configured so as to be capable of receiving liquid from a standard pipette tip.

The sample ports preferably contain a septum (i.e. a partition or dividing wall) which serves as a self-closing inlet to prevent contamination. The septum preferably will open upon contact with a pipette tip, or other instrument used to introduce liquid into the slide, and will close or reseal upon removal of the pipette tip or other such instrument. The septum is preferably constructed of a sealable material such as, for example elastomer, silicone rubber, teflon, etc. As used herein, the term “sealable” means that after introduction of sample, the septum will be able to close and maintain a closed or sealed environment without introduction of unwanted air, liquid, etc. from the outside and without substantial loss of air, fluid, etc. from the inside.

The analysis area may be one open chamber or may be subdivided into any number of smaller subchambers for simultaneous analysis of a variety of different samples using the same slide. Preferably, the subchambers are completely separated so that there is no cross-contamination of samples from one chamber to the next. Each separate subchamber preferably contains its own separate microfluidics system including inlet ports, outlet ports, vents, tubes or channels, etc.

Alternatively, the analysis area may contain one or more extended channels, including an extended channel that traverses repeatedly through the analysis area.

In systems having multiple flow channels, those flow channels may each have separate microfluidic systems (e.g. inlet and outlet ports, waste chambers), or the two or more channels may share a single microfluidic system.

The slides or substrate platforms of the invention may be used for any application which typically utilizes a standard microscope slide. For example, the slides may be used for evaluation of samples such as smears, sections, liquid samples, etc. The samples are preferably applied to the analysis area of the slide. The slides of the invention may be used in conjunction with any type of equipment, instrument or machine typically used to manipulate or evaluate a standard microscope slide.

The slides or substrate platforms of the invention may also be used for binding or immobilizing biomolecules. Biomolecules are preferably bound to the analysis area of the slide. The term ‘biomolecule’ as used herein is meant to indicate any type of nucleic acid, modified nucleic acid, protein, modified protein, peptide, modified peptide, small molecule, lectin, polysaccharide, hormone, drug, drug candidate, etc. Biomolecule binding may be covalent, non-covalent, direct, indirect, via a linker, targeted, random, etc. Biomolecules may be attached through a single attachment to the surface of the substrate platform or via multiple attachments for a single biomolecule. Any type of binding method known to the skilled in the art may be used.

Nucleic acids which may be immobilized onto the substrate include RNA, mRNA, DNA, LNA, PNA, cDNA, oligonucleotides, primers, nucleic acid binding partners, etc. The nucleic acids for immobilization may be modified by any method known in the art. For example, the nucleic acids may contain one or more modified nucleotides, etc. and/or one or more modified intemucleotide linkages, such as, phosphorothioate, etc. Particularly preferred 3′ and/or 5′ modifications include amino modifiers, thiols, and photoreactive ketones particularly quinones, especially anthraquinones.

Particularly preferred modified nucleic acids are those containing one or more nucleoside analogues of the locked nucleoside analogue (LNA) type as described in WO 99/14226, which is incorporated herein by reference. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc.

As disclosed in WO 99/14226, LNA are a novel class of DNA analogues that form DNA- or RNA-heteroduplexes with exceptionally high thermal stability. LNA monomers include bicyclic compounds as shown immediately below:

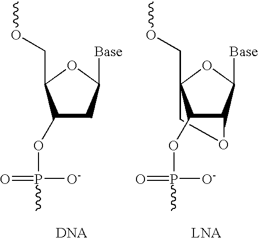

References herein to Locked Nucleoside Analogues, LNA or similar term refers to such compounds as disclosed in WO 99/14226.

LNA monomers and oligomers can share chemical properties of DNA and RNA; they are water soluble, can be separated by agarose gel electrophoresis, can be ethanol precipitated, etc.

Introduction of LNA monomers into either DNA, RNA or pure LNA oligonucleotides results in extremely high thermal stability of duplexes with complimentary DNA or RNA, while at the same time obeying the Watson-Crick base pairing rules. In general, the thermal stability of heteroduplexes is increased 3-8° C. per LNA monomer in the duplex. Oligonucleotides containing LNA can be designed to be substrates for polymerases (e.g. Taq polymerase), and PCR based on LNA primers is more discriminatory towards single base mutations in the template DNA compared to normal DNA-primers (i.e. allele specific PCR). Furthermore, very short LNA oligos (e.g. 8-mers) which have high Tₘ's when compared to similar DNA oligos, can be used as highly specific catching probes with outstanding discriminatory power towards single base mutations (i.e. SNP detection).

Oligonucleotides containing LNA are easily synthesized by standard phosphoramidite chemistry. The flexibility of the phosphoramidite synthesis approach further facilitates the easy production of LNA oligos carrying all types of standard linkers, fluorophores and reporter groups.

Particularly preferred LNA monomer for incorporation into an oligonucleotide for immobilization on the open substrate analysis platform include those of the following formula Ia

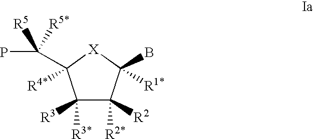

wherein X oxygen, sulfur, nitrogen, substituted nitrogen, carbon and substituted carbon, and preferably is oxygen; B is a nucleobase; R¹*, R², R³, R⁵ and R⁵* are hydrogen; P designates the radical position for an intemucleoside linkage to a succeeding monomer, or a 5′-terninal group, R³* is an intemucleoside linkage to a preceding monomer, or a 3′-terminal group; and R²* and R⁴* together designate —O—CH₂— where the oxygen is attached in the 2′-position, or a linkage of —CH₂)ₙ— where n is 2, 3 or 4, preferably 2, or a linkage of —S—CH₂— or —NH—CH₂—.

Units of formula Ia where R²* and R⁴* contain oxygen are sometimes referred to herein as “oxy-LNA”; units of formula Ia where R²* and R⁴* contain sulfur are sometimes referred to herein as “thio-LNA”; and units of formula Ia where R²* and R⁴* contain nitrogen are sometimes referred to herein as “amino-LNA”. For many applications, oxy-LNA units are preferred modified nucleic acid residues of oligonucleotides of the invention.

As used herein, including with respect to formula Ia, the term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat No. 5,432,272 and Susan M. Freier and Karl-Heinz Altmann, Nucleic Acids Research, 1997, vol. 25, pp 4429-4443. The term “nucleobase” thus includes not only the known purine and pyrimidine heterocycles, but also heterocyclic analogues and tautomers thereof. It should be clear to the person skilled in the art that various nucleobases which previously have been considered “non-naturally occurring” have subsequently been found in nature.

A “non-oxy-LNA” monomer is broadly defined as any nucleoside (i.e. a glycoside of a heterocyclic base) which does not contain an oxygen atom in a 2′-4′-sugar linkage.. Examples of non-oxy-LNA monomers include 2′-deoxynucleotides (DNA) or nucleotides (RNA) or any analogues of these monomers which are not oxy-LNA, such as for example the thio-LNA and amino-LNA described above with respect to formula 1a and in Singh et al. J. Org. Chem. 1998, 6, 6078-9, and the derivatives described in Susan M. Freier and Karl-Heinz Altmann, Nucleic Acids Research, 1997, vol 25, pp 4429-4443.

A wide variety of modified nucleic acids may be employed, including those that have 2′-modification of hydroxyl, 2′-O-methyl, 2′-fluoro, 2′-trifluoromethyl, 2′-O-(2-methoxyethyl), 2′-O-aminopropyl, 2′-O-dimethylamino-oxyethyl, 2′-O-fluoroethyl or 2′-O-propenyl. The nucleic acid may further include a 3′ modification, preferably where the 2′- and 3′-position of the ribose group is linked. The nucleic acid also may contain a modification at the 4′-position, preferably where the 2′- and 4′-positions of the ribose group are linked such as by a 2′-4′ link of —CH₂—S—, —CH₂—NH—, or —CH₂—NMe— bridge.

The nucleotide also may have a variety of configurations such as α-D-ribo, β-D-xylo, or α-L-xylo configuration.

The internucleoside linkages of the residues of oligos of the invention may be natural phosphorodiester linkages, or other linkages such as —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O— PO(NHR^{N})—O—, where R^{H} is selected form hydrogen and C₁₋₄-alkyl, and R″ is selected from C₁₋₆-alkyl and phenyl.

A further preferred group of modified nucleic acids for incorporation into oligomers of the invention include those of the following formula:

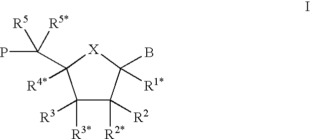

wherein X is —O—; B is selected from nucleobases; R¹* is hydrogen;

P designates the radical position for an intemucleoside linkage to a succeeding monomer, or a 5′-terminal group, such intemucleoside linkage or 5′-terminal group optionally including the substituent R⁵, R⁵ being hydrogen or included in an intemucleoside linkage, R³* is a group P* which designates an internucleoside linkage to a preceding monomer, or a 3′-terminal group;

one or two pairs of non-geminal substituents selected from the present substituents of R², R²*, R³, R⁴*, may designate a biradical consisting of 1-4 groups/atoms selected from —C(R^{a}R^{b})—, —C(R^{a})C(R^{a})—, —C(R^{a})═N—, —O—, —S—, —SO₂—, —N(R^{a})—, and >C═Z,

wherein Z is selected from —O—, —S—, and —N(R^{a})—, and R^{a} and R^{b} each is independently selected from hydrogen, optionally substituted C₁₋₆-alkyl, optionally substituted C₂₋₆-alkenyl, hydroxy, C₁₋₆-alkoxy, C₂₋₆-alkenyloxy, carboxy, C₁₋₆-alkoxycarbonyl, C₁₋₆-alkylcarbonyl, formyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, photochemically active groups, thermochemically active groups, chelating groups, reporter groups, and ligands,

said possible pair of non-geminal substituents thereby forming a monocyclic entity together with (i) the atoms to which said non-geminal substituents are bound and (ii) any intervening atoms; and each of the substituents R², R²*, R³, R⁴* which are present and not involved in the possible biradical is independently selected from hydrogen, optionally substituted C₁₋₆-alkyl, optionally substituted C₂₋₆-alkenyl, hydroxy, C₁₋₆-alkoxy, C₂₋₆-alkenyloxy, carboxy, C₁₋₆-alkoxycarbonyl, C₁₋₆-alkylcarbonyl, formnyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, photochemically active groups, thermochemically active groups, chelating groups, reporter groups, and ligands; and basic salts and acid addition salts thereof.

Particularly preferred LNA monomers for use in the open substrate analysis platform are 2′-deoxyribonucleotides, ribonucleotides, and analogues thereof that are modified at the 2′-position in the ribose, such as 2′-O-methyl, 2′-fluoro, 2′-trifluoromethyl, 2′-O-(2-methoxyethyl), 2′-O-aminopropyl, 2′-O-dimethylamino-oxyethyl, 2′-O-fluoroethyl or 2′-O-propenyl, and analogues wherein the modification involves both the 2′ and 3′ position, preferably such analogues wherein the modifications links the 2′- and 3′-position in the ribose, such as those described in Nielsen et al., J. Chem. Soc., Perkin Trans. 1, 1997, 3423-33, and in WO 99/14226, and analogues wherein the modification involves both the 2′- and 4′-position, preferably such analogues wherein the modifications links the 2′- and 4′-position in the ribose, such as analogues having a —CH₂—S— or a —CH₂—NH— or a —CH₂—NMe— bridge (see Singh et al. J. Org. Chem. 1998, 6, 6078-9). Although LNA monomers having the β-D-ribo configuration are often the most applicable, other configurations also are suitable for purposes of the invention. Of particular use are α-L-ribo, the β-D-xylo and the α-L-xylo configurations (see Beier et al., Science, 1999, 283, 699 and Eschenmoser, Science, 1999, 284, 2118), in particular those having a 2′-4′ —CH₂—S—, —CH₂—NH—, —CH₂—O— or —CH₂—NMe— bridge.

In the present context, the term “oligonucleotide” which is the same as “oligomer” which is the same as “oligo” means a successive chain of nucleoside monomers (i.e. glycosides of heterocyclic bases) connected via intemucleoside linkages. The linkage between two successive monomers in the oligo consist of 2 to 4, preferably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂— CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═ (including R′ when used as a linkage to a succeeding monomer), —S— CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR_{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are especially preferred. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443. The left-hand side of the intemucleoside linkage is bound to the 5-membered ring as substituent P* at the 3′-position, whereas the right-hand side is bound to the 5′-position of a preceding monomer.

The term “succeeding monomer” relates to the neighboring monomer in the 5′-terminal direction and the “preceding monomer” relates to the neighboring monomer in the 3′-terminal direction.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, inosine with C, pseudoisocytosine with G, etc.

An “LNA modified oligonucleotide” is used herein to describe oligonucleotides comprising at least one LNA monomeric residue of the general scheme A, described infra, having the below described illustrative examples of modifications:

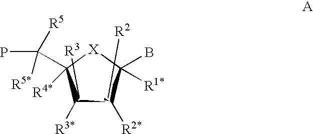

wherein X is selected from —O—, —S—, —N(R^{N})—, —C(R⁶R⁶*)—, —O—C(R⁷R⁷*)—, —C(R⁶R⁶*)—O—, —S—C(R⁷R⁷*)—, —C(R⁶R⁶*)—S—, —N(R^{N}*)—C(R⁷R⁷*)—, —C(R⁶R⁶*)—N(R^{N}*)—, and —C(R⁶R⁶*)—C(R⁷R⁷*)—;

B is selected from hydrogen, hydroxy, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands;

P designates the radical position for an intemucleoside linkage to a succeeding monomer, or a 5′-terminal group, such intemucleoside linkage or 5′-terminal group optionally including the substituent R⁵;

one of the substituents R², R²*, R³, and R³* is a group P* which designates an intemucleoside linkage to a preceding monomer, or a 2′/3′-terminal group;

the substituents of R¹*, R⁴*, R⁵, R⁵*, R⁶, R⁶*, R⁷, R⁷*, R^{N}, and the ones of R², R²*, R³, and R³* not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C═Q,

wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R¹*, R², R²*, R³, R³*, R⁴*, R⁵, R⁵*, R⁶ and R⁶*, R⁷, and R⁷* which are present and not involved in P, P* or the biradical(s) together may form an associated biradical selected from biradicals of the same kind as defined before;

said pair(s) of non-geminal substituents thereby forming a mono- or bicyclic entity together with (i) the atoms to which said non-geminal substituents are bound and (ii) any intervening atoms; and

each of the substituents R¹*, R², R²*, R³, R⁴*, R⁵, R⁵*, R⁶ and R⁶*, R⁷, and R⁷* which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof;

In another preferred embodiment, LNA modified oligonucleotides used in open analysis substrate platform comprises oligonucleotides containing at least one LNA monomeric residue of the general scheme A above:

wherein X, B, P are defined as above;

one of the substituents R², R²*, R³, and R³* is a group P* which designates an internucleoside linkage to a preceding monomer, or a 2′/3′-terminal group;

substituent together designates a biradical structure selected from —(CR*R*)ᵣ—M—(CR*R*)ₛ—, —(CR*R*)ᵣ—M—(CR*R*)ₛ—M—, —M—(CR*R*)ᵣ₊ₛ—M—, —M—(CR*R*)ᵣ—M—(CR*R*)ₛ—,—(CR*R*)ᵣ₊ₛ—, —M—, —M—M—, wherein each M is independently selected from —O—, —S—, —Si(R*)₂—, —N(R*)—, >C═O, —C(═O)—N(R*)—, and —N(R*)—C(═O)—. Each R* and R^{1(1*)}—R^{7(7*)}, which are not involved in the biradical, are independently selected from hydrogen, halogen, azido, cyano, nitro, hydroxy, mercapto, amino, mono- or di(C₁₋₆-alkyl)amino, optionally substituted C₁₋₆-alkoxy, optionally substituted C₁₋₆-alkyl, DNA intercalators, photochemically active groups, thermochemically active groups, chelating groups, reporter groups, and ligands, and/or two adjacent (non-geminal) R* may together designate a double bond, and each of r and s is 0-4 with the proviso that the sum r+s is 1-5.

In a most preferred embodiment LNA-nucleoside conjugates used in the open substrate analysis platform comprise nucleosides containing at least one LNA monomeric residue of the general formula shown scheme B:

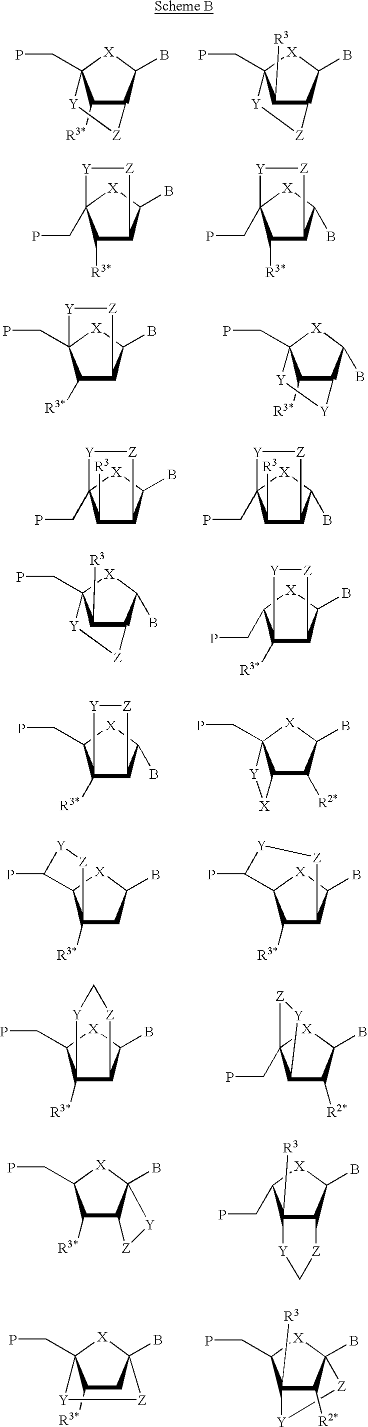

Wherein the groups, X and B are defined as above.

P designates the radical position for an internucleoside linkage to a succeeding monomer, nucleoside such as an L-nucleoside, or a 5′-terminal group, such internucleoside linkage or 5′-terminal group optionally including the substituent R⁵;

one of the substituents R², R²*, R³, and R³* is a group P* which designates an internucleoside linkage to a preceding monomer, or a 2′/3′-terminal group;

Preferred nucleosides are L-nucleosides such as for example, derived dinucleoside monophosphates. The nucleoside can be comprised of either a beta-D, a beta-L or an alpha.-L nucleoside. Preferred nucleosides may be linked as dimers wherein at least one of the nucleosides is a beta-L or alpha-L. B may also designate the pyrimidine bases cytosine, thymine, uracil, or 5-fluorouridine (5-FUdR) other 5-halo compounds, or the purine bases, adenosine, guanosine or inosine.

The chimeric oligos for use in the open substrate analysis platform are highly suitable for a variety of diagnostic purposes such as for the isolation, purification, amplification, detection, identification, quantification, or capture of nucleic acids such as DNA, mRNA or non-protein coding cellular RNAs, such as tRNA, rRNA, snRNA and scRNA, or synthetic nucleic acids, in vivo or in vitro. The use of any of the oligomers described herein, for immobilization onto the open substrate analysis platform allows for a variety of important uses as seen below.

The oligomer can comprise a photochemically active group, a thermochemically active group, a chelating group, a reporter group, or a ligand that facilitates the direct of indirect detection of the oligomer or the immobilization of the oligomer onto a solid support. Such group are typically attached to the oligo when it is intended as a probe for in situ hybridization, in Southern hybridization, Dot blot hybridization, reverse Dot blot hybridization, or in Northern hybridization.

When the photochemically active group, the thermochemically active group, the chelating group, the reporter group, or the ligand includes a spacer (K), the spacer may suitably comprise a chemically cleavable group.

In the present context, the term “photochemically active groups” covers compounds which are able to undergo chemical reactions upon irradiation with light. Illustrative examples of functional groups hereof are quinones, especially 6-methyl-1,4-naphtoquinone, anthraquinone, naphtoquinone, and 1 ,4-dimethyl-anthraquinone, diazirines, aromatic azides, benzophenones, psoralens, diazo compounds, and diazirino compounds.

In the present context “thermochemically reactive group” is defined as a functional group which is able to undergo thernochemically-induced covalent bond formation with other groups. Illustrative examples of functional parts thermochemically reactive groups are carboxylic acids, carboxylic acid esters such as activated esters, carboxylic acid halides such as acid fluorides, acid chlorides, acid bromide, and acid iodides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, and boronic acid derivatives.

In the present context, the term “chelating group” means a molecule that contains more than one binding site and frequently binds to another molecule, atom or ion through more than one binding site at the same time. Examples of functional parts of chelating groups are iminodiacetic acid, nitrilotriacetic acid, ethylenediamine tetraacetic acid (EDTA), aminophosphonic acid, etc.

In the present context, the term “reporter group” means a group which is detectable either by itself or as a part of an detection series. Examples of functional parts of reporter groups are biotin, digoxigenin, fluorescent groups (groups which are able to absorb electromagnetic radiation, e.g light or X-rays, of a certain wavelength, and which subsequently reemits the energy absorbed as radiation of longer wavelength; illustrative examples are dansyl (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO (N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-1-diazole (NBD), pyrene, fluorescein, Europium, Ruthenium, Samarium, and other rare earth metals), radioisotopic labels, chemiluminescence labels (labels that are detectable via the emission of light during a chemical reaction), spin labels (a free radical (e.g. substituted organic nitroxides) or other paramagnetic probes (e.g. Cu²⁺, Mg²⁺) bound to a biological molecule being detectable by the use of electron spin resonance spectroscopy), enzymes (such as peroxidases, alkaline phosphatases, β-galactosidases, and glycose oxidases), antigens, antibodies, haptens (groups which are able to combine with an antibody, but which cannot initiate an immune response by itself, such as peptides and steroid hormones), carrier systems for cell membrane penetration such as: fatty acid residues, steroid moieties (cholesteryl), vitamin A, vitamin D, vitamin E, folic acid peptides for specific receptors, groups for mediating endocytose, epidermal growth factor (EGF), bradykinin, and platelet derived growth factor (PDGF). Especially interesting examples are biotin, fluorescein, Texas Red, rhodamine, dinitrophenyl, digoxigenin, Ruthenium, Europium, Cy5, Cy3, etc.

In the present context “ligand” refers to the binding of a first molecule to another molecule which has an affinity for the first molecule, such as for example a TNF molecule (ligand) binding to the TNF receptor. Ligands can comprise functional groups such as: aromatic groups (such as benzene, pyridine, naphthalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁-C₂₀ alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-α-alanine, polyglycine, polylysine, peptides, oligo/polysaccharides, oligo/polyphosphates, toxins, antibiotics, cell poisons, and steroids, and also “affinity ligands”, i.e. functional groups or biomolecules that have a specific affinity for sites on particular proteins, antibodies, poly- and oligosaccharides, and other biomolecules.

It should be understood that the above-mentioned specific examples under DNA intercalators, photochemically active groups, thermochemically active groups, chelating groups, reporter groups, and ligands correspond to the “active/functional” part of the groups in question. For the person skilled in the art it is furthermore clear that DNA intercalators, photochemically active groups, thermochemically active groups, chelating groups, reporter groups, and ligands are typically represented in the form M-K- where M is the “active/functional” part of the group in question and where K is a spacer through which the “active/functional” part is attached to the 5- or 6-membered ring. Thus, it should be understood that the group B, in the case where B is selected from DNA intercalators, photochemically active groups, thermochemically active groups, chelating groups, reporter groups, and ligands, has the form M-K-, where M is the “active/functional” part of the DNA intercalator, photochemically active group, thermochemically active group, chelating group, reporter group, and ligand, respectively, and where K is an optional spacer comprising 1-50 atoms, preferably 1-30 atoms, in particular 1-15 atoms, between the 5- or 6-membered ring and the “active/functional” part.

In the present context, the term “spacer” means a thermochemically and photochemically non-active distance-making group and is used to join two or more different moieties of the types defined above. Spacers are selected on the basis of a variety of characteristics including their hydrophobicity, hydrophilicity, molecular flexibility and length (e.g see Hermanson et. al., “Immobilized Affinity Ligand Techniques”, Academic Press, San Diego, Calif. (1992), p. 137-ff). Generally, the length of the spacers are less than or about 400 Å, in some applications preferably less than 100 Å. The spacer, thus, comprises a chain of carbon atoms optionally interrupted or terminated with one or more heteroatoms, such as oxygen atoms, nitrogen atoms, and/or sulphur atoms. Thus, the spacer K may comprise one or more amide, ester, amino, ether, and/or thioether functionalities, and optionally aromatic or mono/polyunsaturated hydrocarbons, polyoxyethylene such as polyethylene glycol, oligo/polyamides such as poly-α-alanine, polyglycine, polylysine, and peptides in general, oligosaccharides, oligo/polyphosphates. Moreover the spacer may consist of combined units thereof. The length of the spacer may vary, taking into consideration the desired or necessary positioning and spatial orientation of the “active/functional” part of the group in question in relation to the 5- or 6-membered ring. In particularly interesting embodiments, the spacer includes a chemically cleavable group. Examples of such chemically cleavable groups include disulphide groups cleavable under reductive conditions, peptide fragments cleavable by peptidases, etc.

As discussed above, these oligonucleotides may be used in the open substrate analysis platform for the construction of high specificity oligo arrays e.g. wherein a multitude of different oligos are affixed to a solid surface in a predetermined pattern (Nature Genetics, suppl. vol. 21, January 1999, 1-60 and WO 96/31557). The usefulness of such an array, which can be used to simultaneously analyze a large number of target nucleic acids, depends to a large extend on the specificity of the individual oligos bound to the surface. The target nucleic acids may carry a detectable label or be detected by incubation with suitable detection probes which may also be an oligonucleotide of the invention.

An illustrative example for use of an open substrate analysis platform is for identification of a nucleic acid sequence capable of binding to a biomolecule of interest. This is achieved by immobilizing a library of nucleic acids onto the substrate surface so that each unique nucleic acid is located at a defined position to form an array. The array is then exposed to the biomolecule under conditions which favor binding of the biomolecule to the nucleic acids. Non-specifically binding biomolecules are washed away using mild to stringent buffer conditions depending on the level of specificity of binding desired. The nucleic acid array is then analyzed to determine which nucleic acid sequences bound to the biomolecule. Preferably the biomolecules would carry a fluorescent tag for use in detection of the location of the bound nucleic acids.

The open substrate platforms, with an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Nucleic acids for immobilization onto the substrate may be either single stranded or double stranded and preferably contain from about 2 to about 1000 nucleotides, more preferably from about to 2 to about 100 nucleotides and most preferably from about 2 to about 30 nucleotides.

Polypeptides may also be immobilized onto the surface of the substrate platform. Particularly preferred polypeptides for immobilization are receptors, ligands, antibodies, antigens, enzymes, nucleic acid binding proteins, etc. Polypeptides may be modified in any way known to those skilled in the art. For example, polypeptides may contain one or more phosphorylations, glycosylations, etc. Additionally, polypeptides may be attached to a flexible linker and/or reactive to group to facilitate binding to the surface of the substrate.

Polypeptides for immobilization onto the substrate may be monomeric, dimeric or multimeric and preferably contain from about 2 to about 1000 amino acids, more preferably from about 2 to about 100 amino acids and most preferably from about 2 to about 20 amino acids.

Polypeptides and nucleic acids for immobilization onto the substrate may be prepared separately and then applied onto the substrate surface. Methods for preparation of nucleic acids/oligos are known in the art, for example phosphoramidite chemistry.

Polypeptides and nucleic acids may be applied to the surface of the substrate by any method well known in the art. For example, polypeptides or nucleic acids may be manually pipetted onto the surface or applied using a robotics system. Preferably, polypeptides or nucleic acids are applied to the substrate using a micro spotting technique such as may be achieved with inkjet type technology.

The analysis substrates of the invention also may be employed for relatively high density analysis, e.g. loaded for analysis with at least about 100 unique polypeptide sequences or nucleotides sequences per cm²of analysis area; or at least about 200, 300, 400, 500, 600, 700, 800 or 900 unique polypeptide sequences or nucleotides sequences per cm²of analysis area.

Biomolecules may be attached to the surface of the substrate using any method known in the art. Preferably biomolecules are attached to the surface using a photochemical linker which becomes active upon exposure to light of a defined wavelength. Most preferably biomolecules are attached to the surface using a quinone photolinker. Methods for photochemical immobilization of biomolecules using quinones are described in WO 96/31557, which is incorporated herein by reference.

Biomolecules may be attached directly to the analysis substrate surface or may be attached to the substrate through a flexible linker group. The linker group may be attached to the surface of the substrate before immobilization of the biomolecule or the linker group may be attached to the biomolecule before immobilization onto the substrate. For example, a nucleic acid may be modified with a linker group at either the 3′ or 5′ end prior to immobilization onto the substrate. Alternatively, an unmodified nucleic acid may be attached to the substrate which has been coated with linker groups. Similarly, a polypeptide may be modified with a group at either the amino terminus or carboxy terminus prior to immobilization onto the substrate. Alternatively, an unmodified polypeptide may be immobilized onto the substrate which has been coated with linker groups. The linker groups may be attached at any location within a nucleic acid or polypeptide chain but are preferably attached at either end of the polypeptide or amino acid chain. Linker groups for immobilization of biomolecules are well known in the art. Any linker group known in the art may be used for attachment of biomolecules.

Alternatively, polypeptides and nucleic acids may be synthesized in situ on the surface of the substrate. Methods for in situ synthesis of polypeptides and nucleic acids are well known in the art and include photolithographic techniques, protection/deprotection techniques, etc.

The analysis area of the substrate platforms of the invention may be coated with a single biomolecule, with a random mixture of biomolecules or with a mixture of biomolecules wherein each unique biomolecule is located at a defined position so as to form an array. In a preferred embodiment the analysis area is coated with a library of polypeptides or nucleic acids wherein each unique nucleic acid or amino acid sequence is located at a defined location within the analysis area.

The invention also provides methods for using the substrate platforms of the invention for carrying out a variety of bioassays. Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

Identification of a nucleic acid sequence capable of binding to a biomolecule of interest could be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analysed to determine which nucleic acid sequences bound to the biomolecule. Preferably the biomolecules would carry a fluorescent tag for use in detection of the location of the bound nucleic acids.

Assay using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Assays using immobilized polypeptides are also provided by the methods of the invention. For example, an immobilized array of peptides could be exposed to an antibody or receptor to determine which peptides are recognized by the antibody or receptor. Preferably the antibody or receptor carriers a fluorescent tag for identification of the location of the bound peptides. Alternatively, an immobilized array of antibodies or receptors could be exposed to a polypeptide to determine which antibodies recognize the polypeptide.

The slides of the invention may also be used for assays not involving immobilised biomolecules. For example, the slides may be used for cell sorting, including living cells (inclusive of viruses), which sorted cells then may be subjected to analysis.

Analysis substrates of the invention also may be modified as appropriate for particular assays. For instance, in closed analysis systems of the invention, one or more surfaces of the internal analysis surface can be pre-treated to facilitate attachment and/or growth of cells for analysis.

All documents mentioned herein are incorporated herein by reference in their entirety.

The invention has been described in detail with reference to preferred embodiments thereof. However, it will be appreciated that those skilled in the art, upon consideration of this disclosure, may make modifications and improvements within the spirit and scope of the invention.

What is claimed is:

1. An open substrate platform comprising:
a slide element having opposing top and bottom surfaces,
wherein the top surface of the slide contains one or more depressions with a defined area for sample analysis,
wherein the bottom surface of the slide contains one or more depressions opposing the depression on the top surface, and
wherein the bottom surface of the slide further comprises at least one set of paired finger indentations for use in removing the slide from a flat surface.

2. The open substrate platform of claim 1, further comprising a coverslip,
wherein the coverslip is capable of the analysis area on the top surface of the slide, and
wherein the coverslip is constructed of a material which is more hydrophilic than the material from which the slide is constructed.

3. The open substrate platform of claim 1, further comprising a coverslip,
wherein the coverslip is capable of the analysis area on the top surface of the slide, and
wherein the coverslip is constructed of a material which has the same hydrophilicity as the material from which the slide is constructed.

4. An open substrate platform comprising:
a slide element having opposing top and bottom surfaces, the slide element preferably being substantially rectangular and formed from a plastic material, and
wherein the top surface of the slide is comprised of a defined area for sample analysis, and
wherein the bottom surface of the slide contains one or more depressions; wherein, the bottom surface of the slide further comprises at least one set of paired finger indentations for use in removing the slide from a flat surface.

5. The substrate platform of claims 1 or 4, wherein the substrate platform further comprises a mark used to identify the slide.

6. The substrate platform of claim 5, wherein the mark is a bar code.

7. The substrate platform of claims 1 or 4, wherein the slide is constructed from one or more materials of polycarbonate or Topas.

8. The substrate platform of claims 1 or 4, wherein the slide is constructed from plastic.

9. The substrate platform of claims 1 or 4, wherein the slide has a flatness of less than or equal to about 20 μm, wherein the flatness does not deviate on a slide and between slides, more than 1 μm per millimeter.

10. The substrate platform of claims 1 or 4, wherein the slide has a roughness of about an RA of less than about 100 nm.

11. The substrate platform of claims 1 or 4, wherein the slide has a roughness with an RA of less than about 50 nm.

12. The substrate platform of claims 1 or 4, wherein the slide has a roughness with an RA of less than about 20 nm.

13. The substrate platform of claims 1 or 4, wherein the surface of the slide is treated so as to increase the binding capacity of the slide.

14. The substrate platform of claims 1 or 4, wherein the slide is constructed of a material which is resistant to temperatures over a range of −5° C. to +105° C.

15. The substrate platform of claims 1 or 4, wherein the slide is constructed of a material which is resistant to pH over a range of pH=1 to pH=13.

16. The substrate platform of claims 1 or 4, which is dimensioned so as to be compatible with equipment capable of handling a standard microscope slide.

17. The substrate platform of claims 1 or 4, which is constructed using injection molding.

19. The substrate platform of claims 1 or 4, which further comprises immobilized nucleic acid sequences.

20. The substrate platform of claim 19, wherein the nucleic acid sequences are modified.

21. The substrate platform of claim 20, wherein the nucleic acid sequences contain at least one modified nucleotide.

22. The substrate platform of claim 20, wherein the nucleic acid sequences contain at least one locked nucleoside analogue.

24. The substrate platform of claim 20, wherein the nucleic acid sequences are completely composed of locked nucleoside analogues.

25. The substrate platform of claim 20, wherein the nucleic acid sequences contain at least one modified internucleoside linkage.

26. The substrate platform of claim 20, wherein the nucleic acid sequences contain at least one phosphorothioate intemucleoside linkage.

27. The substrate platform of claim 20, wherein all of the internucleoside linkages of the nucleic acid sequences are phosphorothioate.

28. The substrate platform of claim 20, wherein the nucleic acid sequences comprise at least one modified nucleotide and at least on modified internucleoside linkage.

29. The substrate platform of claim 19, wherein each immobilized nucleic acid with a unique sequence is located at a defined position.

30. The substrate platform of claim 29, which comprises at least 100 unique sequences per cm².

31. The substrate platform of claim 29, which comprises at least 400 unique sequences per cm².

32. The substrate platform of claim 29, which comprises at least 900 unique sequences per cm².

33. The substrate platform of claim 29, wherein each immobilized nucleic acid contains from about 500 to about 1000 nucleotides.

34. The substrate platform of claim 29, wherein each immobilized nucleic acid contains from about 100 to about 500 nucleotides.

35. The substrate platform of claim 29, wherein each immobilized nucleic acid contains from about 10 to about 100 nucleotides.

36. The substrate platform of claim 29, wherein each immobilized nucleic acid contains from about 2 to about 30 nucleotides.

37. The substrate platform of claim 19, wherein the nucleic acid sequences are immobilized onto the slide using a photochemical linker.

38. The substrate platform of claim 37, wherein the nucleic acid sequences are immobilized onto the slide using anthraquinone.

39. The substrate platform of claim 19, wherein a linker connects either the 5′ or 3′ ends of the nucleic acid sequences to the surface of the slide.

40. The substrate platform of claim 19, wherein the nucleic acid sequences are immobilized onto the surface of the slide after synthesis.

41. The substrate platform of claim 19, wherein the nucleic acid sequences are synthesized on the surface of the slide.

42. The substrate platform of claim 19, wherein the nucleic acid sequences are double stranded.

43. The substrate platform of claim 19, wherein the nucleic acid sequences are single stranded.

44. The substrate platform of claims 1 or 4, which further comprises immobilized polypeptides.

45. The substrate platform of claim 44, wherein the immobilized polypeptides contains at least one modification selected from the group consisting of phosphorylation or glycosylation.

46. The substrate platform of claim 44, wherein each immobilized polypeptide with a different amino acid sequence is located at a defined position.

47. The substrate platform of claim 46, which comprises at least 100 unique polypeptide sequences per cm².

48. The substrate platform of claim 46, which comprises at least 400 unique polypeptide sequences per cm².

49. The substrate platform of claim 46, which comprises at least 900 unique polypeptide sequences per cm².

50. The substrate platform of claim 44, wherein the polypeptides are immobilized onto the slide using a photochemical linker.

51. The substrate platform of claim 44, wherein the polypeptides are immobilized onto the slide using anthraquinone.

52. The substrate platform of claim 44, wherein a flexible linker connects either the amino-termini or carboxy-termini of the polypeptides to the surface of the slide.

53. The substrate platform of claim 44, wherein the polypeptides are synthesized on the surface of the slide.

54. The substrate platform of claim 1 or 4, wherein the analysis area is modified to facilitate attachment and growth of cells.

55. A method for identifying a nucleic acid sequence capable of binding to a biomolecule comprising:
immobilizing each unique nucleic acid sequence from a library of nucleic acid sequences onto the substrate platform of claims 1 or 4,
optionally washing the substrate platform to remove contaminants,
incubating the immobilized nucleic acid sequences with a biomolecule under conditions which are conducive to specific interaction between the biomolecule and the nucleic acid sequences,
optionally washing the substrate platform to remove any non-specifically bound biomolecules,
detecting the location of the nucleic acid sequences which bound to the biomolecule.

56. The method of claim 55, wherein the biomolecule is a nucleic acid sequence.

57. The method of claim 55, wherein the biomolecule is a polypeptide.

58. The method of claim 55, wherein the location of the nucleic acid sequences which bound to the biomolecule is detected by virtue of a tag on the biomolecule.

59. The method of claim 58, wherein the tag on the biomolecule is a detectable moiety.

60. A method for identifying a polypeptide capable of binding to a biomolecule comprising:
immobilizing each unique polypeptide from a library of polypeptides onto the substrate platform of claims 1 or 4,
optionally washing the substrate platform to remove contaminants,
incubating the immobilized polypeptides with a biomolecule under conditions which are conducive to specific interaction between the biomolecule and the polypeptides,
optionally washing the substrate platform to remove any non-specifically bound biomolecules,
detecting the location of the polypeptides which bound to the biomolecule.

61. The method of claim 60, wherein the biomolecule is a nucleic acid sequence.

62. The method of claim 61, wherein the biomolecule is a polypeptide.

63. The method of claim 61, wherein the biomolecule is a multimeric polypeptide.

64. The method of claim 61, wherein the biomolecule is an antibody.

65. The method of claim 61, wherein the biomolecule is a receptor.

66. The method of claim 61, wherein the biomolecule is a hormone.

67. The method of claim 61, wherein the biomolecule is a drug or drug candidate.

68. The method of claim 61, wherein the location of the polypeptides which bound to the biomolecule is detected by virtue of a tag on the biomolecule.

69. The method of claim 68, wherein the tag on the biomolecule is a fluorescent tag.

70. A method for sample analysis comprising:
applying a sample to the substrate platform of claims 1 or 4; and
evaluating the sample.

71. The method of claim 70, wherein the sample is a liquid.

72. The method of claim 70, wherein the sample is a solid.

73. Use of the substrate platform of claims 1 or 4 for sample analysis.
